(19) United States
(12) Patent Application Publication
(10) Pub. No.: US 2005/0085909 A1
(43) Pub. Date: Apr. 21, 2005

(54) Semi-constrained and mobile-bearing disc prosthesis

(75) Inventor: Lukas Eisermann, Memphis, TN (US)

Correspondence Address: HAYNES AND BOONE, LLP, 901 MAIN STREET, SUITE 3100, DALLAS, TX, 75202, US

(73) Assignee: SDGI Holding, Inc., Wilmington, DE (US)

(21) Appl. No.: 10/685,745

(22) Filed: Oct. 15, 2003

(51) Int. Cl. A61F 2/44

(52) U.S. Cl. 623/17.11

(57) ABSTRACT

A disc replacement device is provided for replacing a spinal disc in an animal subject. The disc replacement device includes a shell, a fulcrum, and a damping sleeve. The shell further comprises a first portion adapted for articulating with the fulcrum such as a spherical stainless steel ball, and a second portion adapted for coupling with the damping sleeve.

### FIELD OF THE INVENTION

The present invention relates generally to artificial replacement devices, and more particularly, to artificial disc replacement devices.

### BACKGROUND

Intervertebral discs are located between the concave articular surfaces of the adjacent vertebral body endplates. They form important and unique articulating systems in the spine, allowing for multiplanar motion. In general, they permit movements such as flexion, extension, lateral flexion, and rotation.

However, the intervertebral disc often experiences anatomical changes, such as disc degeneration, with advancing age. The most significant changes to the disc include: the water and proteoglycan content of the nucleus pulposus decreases; the water proteoglycan content of the annulus decreases, but not the extent of the nucleus; the collagen fibers of the annulus fibrosus become distorted; and tears occur in the lamellae, due in part to distortion of the collagen fibers, one or more of which could result in a loss of annular strength.

As a result of those changes, the disc begins to lose normal height and volume. It becomes progressively less resistant and resilient to loading forces. The nucleus loses the ability to sustain hydrostatic pressure and deform properly because of water loss and because the annular fibers can no longer maintain tension of its web-like lattice structure. In essence, the disc no longer fully acts like a shock absorber between the vertebral bodies. More axial load is then transferred from the central nucleus to the peripheral annulus, resulting in anatomical changes to the vertebral endplates, bodies, and facets. Narrowing the disc space also causes instability in the segment, which in turn adds additional stresses to the other components, particularly the ligaments.

Disc replacement devices have been used to replace injured or damaged intervertebral discs. However, previous disc replacement devices possess a number of disadvantages. For example, one prior art solution is to place a steel ball (commonly referred to as the “Fernstrom ball”) between the vertebrae to maintain an appropriate height between the vertebrae. However, over time, the steel ball tends to migrate into adjacent vertebrae, causing unintended damage.

Therefore, it is desired to provide a more stable disc replacement device to treat a wide range of disorders, and/or provide better pain relief to an animal subject.

### SUMMARY

The present invention provides an enhanced disc replacement device for replacing a spinal disc between two vertebral bodies of the spine. It is directed to solving a number of the problems existing in the previous disc replacement devices.

In one embodiment, a disc replacement device comprises a shell, a fulcrum, and a damping sleeve. The fulcrum can be, in some embodiments, a stainless steel ball, such as a Fernstrom ball. The shell further comprises a first portion adapted for articulating with the fulcrum and a second portion adapted for coupling with the damping sleeve.

In another embodiment, a shell system is provided for use with a spherical ball bearing disc replacement device, such as a Fernstrom ball. The shell system includes a first shell comprising a first portion adapted for coupling with a second shell and a second portion adapted for coupling with a damping sleeve, and a second shell comprising a first surface adapted for coupling with the first portion of the first shell and a second surface adapted for articulating with the spherical ball bearing.

In a third embodiment, a disc replacement device comprises a first shell comprising an opening and an inner surface portion, a pillar adapted for coupling with the first shell at the opening, and a damping sleeve for coupling with the first shell at the inner surface.

### BRIEF DESCRIPTION OF THE DRAWINGS

FIG. 1A is a perspective view of a disc replacement device according to one embodiment of the present invention.

FIG. 1B is a cross-sectional view of a disc replacement device according to one embodiment of the present invention.

FIG. 2 is a cross-sectional view of a disc replacement device shell according to one embodiment of the present invention.

FIG. 3 is a cross-sectional view of a disc replacement device shell according to one embodiment of the present invention.

FIG. 4 is a top view of a disc replacement device according to one embodiment of the present invention.

FIG. 5 is a cross-sectional view of a disc replacement device according to one embodiment of the present invention.

FIG. 6 is a cross-sectional view of a partial disc replacement device according to one embodiment of the present invention.

FIG. 7 is an assembled cross-sectional view of a partial disc replacement device of FIG. 6.

FIG. 8 is a cross-sectional view of a partial disc replacement device according to one embodiment of the present invention.

FIG. 9 illustrates two disc replacement devices residing in a disc space between two adjacent vertebral bodies according to one embodiment of the present invention.

FIG. 10 is a side view of the disc replacement devices and the disc space of FIG. 9.

FIG. 11 is a partial view of a disc space, a disc replacement device and an insertion device according to one embodiment of the present invention.

### DETAILED DESCRIPTION

For the purposes of promoting an understanding of the principles of the invention, references will now be made to the embodiments, or examples, illustrated in the drawings, and specific languages will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

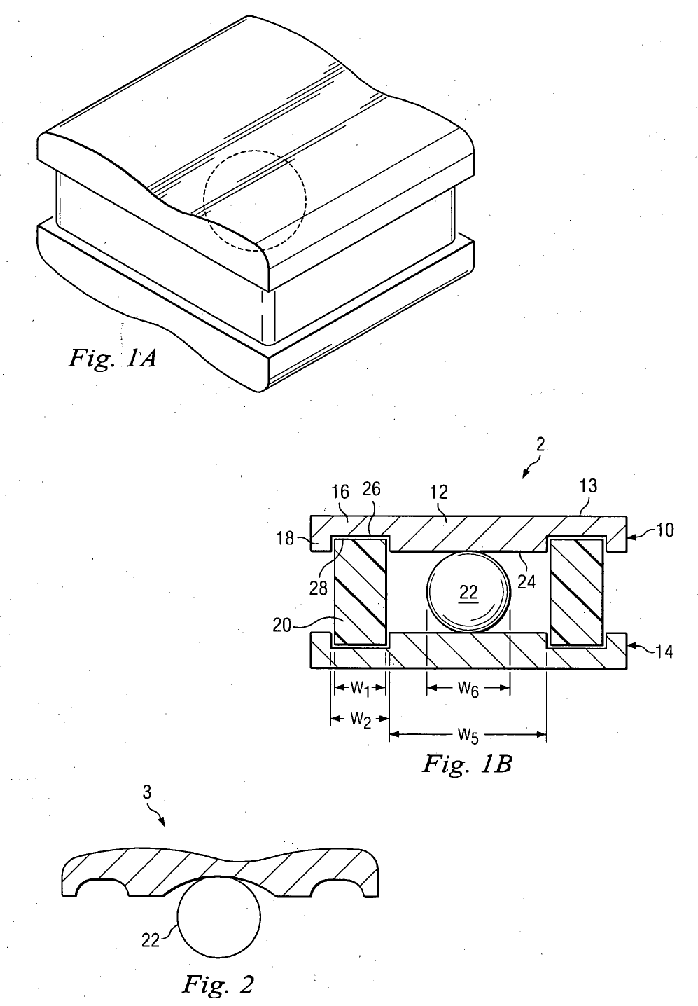

The present invention provides an improved disc replacement device for replacing a disc in an animal subject. In one example, the present invention takes advantage of the simple and elegant Fernstrom ball, and improves its structure by providing bearing surfaces against the vertebral endplates. In many of the embodiments described below, a single bearing surface is shown. However, it will be understood that any of the two bearing surfaces (alternatively, “shells”) may be used to form a set of upper and lower shells. FIG. 1A shows a perspective view of an exemplary disc replacement device.

Referring now to FIG. 1B, shown therein is a disc replacement device 2 according to one embodiment of the present invention. The disc replacement device 2 includes an upper shell 10, a lower shell 14, a spherical ball bearing 22, and a damping sleeve 20.

In the illustrated embodiment, the upper shell 10 and the lower shell 14 are substantially identical. However, it is contemplated that without deviating from the spirit and scope of the present invention, there could be differences between the upper shell 10 and the lower shell 14. Further, each of the embodiments of the shells described in the present invention may be used as an upper shell and/or a lower shell of a disc replacement device. Therefore, identical or different shells may be combined to form the upper and lower shells of a disc replacement device. In the description that follows, the description of the upper shell 10 applies with like effect to the lower shell 14.

In this illustration, an outer surface 13 of the upper shell 10 is shown to be flat. However, it is contemplated, that the outer surface 13 may comprise a variety of other shapes, such as a cylindrical, partial cylindrical, partial spherical or spherical shape, to facilitate the mating of the disc replacement device 2 with an endplate of a vertebra. Likewise, each outer surface of the shells to be described in the present invention may comprise a variety of shapes, such as a flat, cylindrical, partial cylindrical, partial spherical, or spherical shape.

In this example, the upper shell 10 may comprise a first portion 12 and a second portion 16. The first portion 12 may be adapted for articulating with the spherical ball bearing 22, and the second portion may be adapted for coupling with a damping sleeve 20. It is also contemplated that the upper shell 10 may comprise a closure portion 18, which may partially shield the damping sleeve 20.

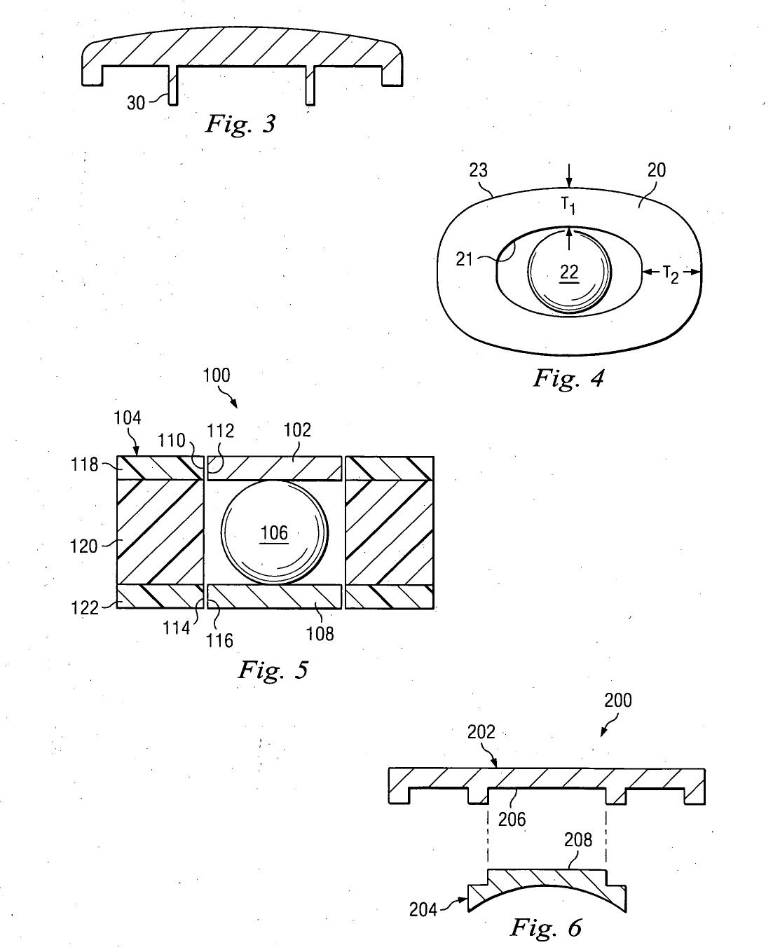

In this illustration, the first portion 12 has an inner surface 24 for articulating with the spherical ball bearing 22. In one example, the first portion may be flat to enable the free movement of the spherical ball bearing 22. However, a variety of other shapes that may enable (or impede) the movement of the spherical ball bearing 22 are also contemplated. For example, the shape of the inner surface 24 may be concaved (as illustrated in FIG. 2), cylindrical, partially cylindrical, spherical, partially spherical, or irregular. It is also contemplated that the first portion 12 may comprise a smooth surface to allow the free movement of the spherical ball bearing 22. Alternatively, it may comprise a rough surface or restrict the movement of the spherical ball bearing 22, so that the spherical ball bearing 22 may not glide and pivot freely. It is contemplated that a width W5 may be larger than or nearly identical to a diameter W6 of the spherical ball bearing 22, so that the spherical ball bearing 22 may or may not move freely. In addition, it is contemplated that the first portion 12 may comprise other features. For example, as illustrated in FIG. 3, the first portion 12 may comprise an internal ring 30 of any shape, which may prevent the spherical ball bearing 22 from exerting excessive force against the damping sleeve 20.

In this illustration, from a cross-sectional view, the second portion 16 has an inner surface 26 that is a partial rectangular for mating with the damping sleeve 20. However, it is contemplated that the inner surface 26 may comprise a variety of other shapes, such as a partial circular (as illustrated in FIG. 2), oval, flat or irregular shape.

In furtherance of this example, the damping sleeve 20 is coupled with the second portion 16. The damping sleeve 20 may serve to prevent the spherical ball bearing 22 from moving too far from its designed position and becoming dislocated. In addition, the damping sleeve 20, may provide various degrees of flexibility to the disc, replacement device, and modulates the stiffness of the disc replacement device 2. The damping sleeve 20 may be mated with the second portion 16 in a variety of means. In one example, the damping sleeve 20 may be bonded to the second portion 16. In another example, the damping sleeve 20 may simply be coupled with the second portion 16. In yet another example, the damping sleeve 20 may contain a cavity, which may comprise any biocompatible lubrication medium, such as hydrogel, silicone, polyurethane or collagen. In yet another example, the damping sleeve 20 may include a lip, which could be a “boss” that internally abuts the damping sleeve 20, or a chamfered or rounded (or otherwise treated) edge to provide certain internal backing, so that the damping sleeve 20 is not purely resisting the shear load. In yet another example, a series of interdigitating pegs (“fingers”) may protrude from the damping sleeve 20 (or the upper shell 10 or the lower shell 14). Those fingers may be in the range of 1 mm in diameter by 1-2 mm in length, or in any other smaller or larger sizes.

In this example, from a cross-sectional view, the damping sleeve 20 has an inner surface 28 that may comprise a variety of shapes, such as a partial rectangular, partial circular, oval, flat or irregular, shape.

In furtherance of this example, the damping sleeve 20 has a width W1, while the second portion 16 has a width W2. To accommodate the movement of a spine, the width W2 may be larger than the width W1 to allow mobility of the damping sleeve 20 relative to the second portion 16. However, it is also contemplated that the width W2 may be nearly identical to the width W1, so that a tight fit may be provided between the damping sleeve 20 and the second portion 16.

The upper shell 10 may comprise any biocompatible material, such as 22Co-13Cr-5Mo, cobalt chrome, stainless steel, titanium, shape memory alloys, polymers, carbon fiber, polyethylene, porous material, silicone, or any orthopedic articular bearing material. Likewise, the damping sleeve 20 may comprise a variety of biocompatible materials, such as silicone, polyurethane, elastomer, polymer or shape memory alloys. It is also contemplated that in selecting a material for the upper shell 10, the materials used for the spherical ball bearing 22 and the damping sleeve 22 may be taken into consideration.

Referring now to FIG. 4, in one embodiment, the damping sleeve 20 may vary in its thickness to provide flexibility and support suitable for different regions of the vertebral endplates. For example, a thickness T1 may be smaller than a thickness T2. However, it is also contemplated that the damping sleeve 20 may comprise a consistent thickness in its entirety. Further, in this illustrated top view, the damping sleeve 20 comprises a donut shape. However, it is contemplated that a variety of other shapes are contemplated. For example, each of the surfaces 21 and 23 may comprise a rectangular, partial rectangular, circle, partial circle, annular disc or irregular shape. It is also contemplated that the surfaces 21 and 23 may have identical (with different sizes) or different shapes. It is further contemplated that instead of a continuous piece as illustrated here, the damping sleeve 20 may comprise a plurality of components.

Referring back to FIG. 1B, in one embodiment, the spherical ball bearing 22 may simply be a biocompatible ball such as a Fernstrom ball. The spherical ball bearing 22 provides mobility similar to that of a natural disc, offers separation of the adjacent vertebrae, and maintains an appropriate height for the disc space. In one embodiment (as shown in FIG. 2), the spherical ball bearing 22 may not translate against a shell 3. However, it is contemplated that the spherical ball bearing 22 may translate against adjacent shells in other embodiments. The spherical ball bearing 22 may comprise any biocompatible material, such as stainless steel, polymer, polyethylene, synthetic diamond, or composite materials. It will be understood that the Fernstrom ball is known in the art, and will not be further described herein.

Referring now to FIG. 5, in another embodiment, the arrangement of FIG. 1B may be modified to form a disc replacement device 100, which may comprise an upper shell 102, a ball 106, a lower shell 108, and a damping sleeve 104. Here, a side surface 110 of the damping sleeve 104 is coupled with a side surface 112 of the upper shell 102, and a side surface 114 of the damping sleeve 104 is coupled with a side surface 116 of the lower shell 108.

In this example, the damping sleeve 104 may comprise a top portion 118, a body 120, and a lower portion 122, and those components of the damping sleeve 104 may be formed together in a variety of means. In one example, the damping sleeve 104 may be a single-piece device comprising the top and lower portions 118, 122, and the body 120. In a second example, the body 120 may be bonded to the top and lower portions 118 and 122. In a third example, the body 120 may simply be coupled with the top and lower portions 118 and 122. In a fourth example, the body 120 (and optionally the top and/or lower portions 118 and 122) may contain a cavity, which may comprise any biocompatible lubrication medium, such as hydrogel, silicone, polyurethane or collagen. In a fifth example, the damping sleeve 104 may include a lip, which could be a “boss” that internally abuts the damping sleeve 104, or a chamfered or rounded (or otherwise treated) edge to provide certain internal backing, so that the damping sleeve 104 is not purely resisting the shear load. In a sixth example, a series of interdigitating pegs (“fingers”) may protrude from the damping sleeve 104 (or the upper shell 102 or the lower shell 108). Those fingers may be in the range of 1 mm in diameter by 1-2 mm in length, or in any other smaller or larger sizes.

It is contemplated that the damping sleeve 104 may comprise a variety of biocompatible materials, such as silicone, polyethylene or shape memory alloys.

Similar to the descriptions with respect to FIGS. 1-3, the upper shell 102 and/or the lower shell 108 may comprise a variety of shapes, such as a concaved (as illustrated in FIG. 2), cylindrical, partial cylindrical, spherical, partially spherical, or irregular shape. It is also contemplated that the upper shell 102 and/or the lower shell 108 may comprise a smooth surface to allow free movement of the ball 106. Alternatively, the upper shell 102 and/or the lower shell 108 may comprise a rough surface, so that the ball. 106 may not glide freely. It is further contemplated that the upper shell 102 and/or the lower shell 106 may comprise other features. For example, an inner ring (as illustrated in FIG. 3) or similar structures may be added to prevent the ball 106 from moving out of its desired range of positions, and/or to mitigate the force exerted by the ball 106 on the. damping sleeve 104.

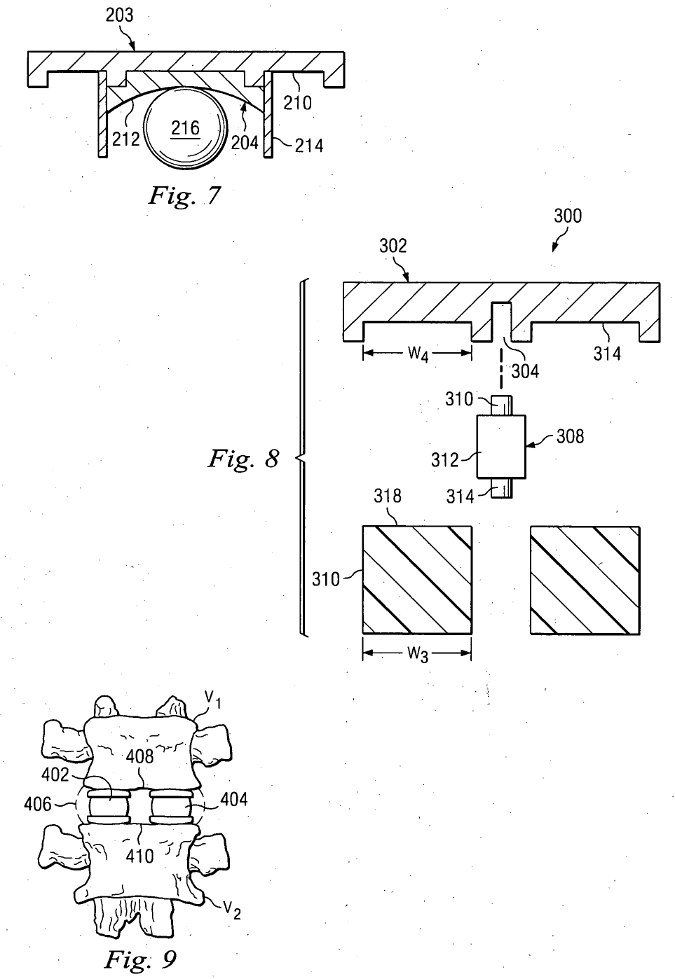

Referring now to FIG. 6, in yet another embodiment, the upper shell 10 and/or the lower shell 14 of FIG. 1B may be modified to form a shell 200, which comprises a first shell 202 and a second shell 204. An inner surface 206 of the first shell 202 may be coupled with a surface 208 of the second shell 204 to form an upper shell 203 of FIG. 7 (or a lower shell of a disc replacement device). The first and second shells 202 and 204 may be coupled by a variety of means. For example, the coupling may be tight or bonded, so that relative movement between the two pieces will be minimized. Alternatively, the coupling may be loose, so that the first shell 202 and the second shell 204 may be allowed to translate with respect to each other, thereby mitigating forces created during a spinal movement. From the cross-sectional view, it will be understood that each of the coupling surfaces 206 and 207 may comprise a variety of shapes, such as a circular, partially circular, or irregular shape.

In furtherance of this example, the first shell 202 may comprise any biocompatible material that may enhance its compatibility with bones or facilitate easy imaging processing, such as 22Co-13Cr-5Mo, titanium, cobalt chrome, stainless steel, polymer, carbon fiber or polythene. The second shell 204 may comprise any biocompatible material, which may be durable and compatible with the composition of the ball 216, such as ceramic, cobalt chrome, stainless steel, or polyethylene. However, it should be understood that each of the first shell 202 and the second shell 204 may comprise any biocompatible implant-grade material, including but not limited to the materials listed above.

Referring now to FIG. 7, in furtherance of this example, the shell 202 may comprise a second portion 210 that is adapted for coupling with a damping sleeve as described with respect to FIG. 1B. Further, as described previously with respect to FIGS. 1-3, from a cross-sectional view, the surface 212 may comprise a variety of shapes, such as a partial rectangular (as shown in FIG. 1B), a partial circular (as illustrated in FIG. 2), oval, flat or irregular shape. It is also contemplated that the surface 212 may comprise a smooth region to allow free movement of the ball 216. Alternatively, it may comprise a rough region, so that the ball 216 may not glide freely.

It is contemplated that the shell 203 may comprise other features. For example, an inner ring 214 may be added to prevent the ball 216 from moving out of its desired range of positions, and/or to mitigate the force exerted by the ball 216 on the damping sleeve.

It is also contemplated that the two-piece arrangement associated with FIGS. 6-7 may also be utilized in the embodiments associated with FIG. 5, so that the upper shell 102 and/or lower shell 108 of FIG. 5 may comprise an identical or similar two-piece arrangements.

Referring now to FIG. 8, shown therein is a disc replacement device 300 according to yet another embodiment of the present invention. The disc replacement device 300 includes a shell 302, a pillar 308 and a damping sleeve 310. In this example, it is understood that the shell 302 may serve as an upper shell or a lower shell, and that the upper shell may be identical to or different from the lower shell.

In furtherance of the example, the pillar 308 comprises a tip 310, a body 312 and a tip 314. Each of the tips 310 and 314 may be adapted to couple with an opening of an upper or lower shell. For example, the tip 310 may be adapted for mating with an opening 304 of the shell 302. It will be understood that descriptions of the tip 310 below shall also apply with like effect to the tip 314.

In this illustration, the tip 310 is substantially cylindrical, and couples with the opening 304 that is also substantially cylindrical. However, it is contemplated that the tip 310 may comprise other conventional or unconventional shapes, such as a polygon, partial rectangular prism, cube, partial cube, sphere, partial sphere, pyramid, partial pyramid, cone, partial cone, or an irregular shape. Similarly, the opening 304 may also comprise any of those shapes to mate with the tip 310.

The body 308 may comprise a super elastic (shape memory) material, such as Nitinol or copper-based alloys, to perform functions of a disc. For example, the body 308 may allow the occurrence of the spinal bending motions. The tips 310 and/or 314 may also comprise a super elastic (shape memory) material, such as. Nitinol or copper-based alloys, and may assist the body 308 in performing disc functions. It is contemplated that the body body 308 and the tips 310, 314 may be produced from a single-piece material. Alternatively, they may be created by separate pieces of materials, and connected together by any conventional means, such as by bonding or being screwed together. In that case, it is contemplate that the tips 310 and/or 314 may comprise any biocompatible material, such as 22Co-13Cr-5Mo, cobalt chrome, stainless steel, titanium, shape memory alloys, polymers, carbon fiber, polythene, polyurethane, polyethylene, porous material or silicone.

In furtherance of this example, the shell 302 comprises the opening 304 for coupling with the pillar 310, and an inner surface portion 314 for coupling with a damping sleeve 310. From a cross-sectional view, the inner surface portion 314 is a partial rectangular adapted to mate with the damping sleeve 310. However, it is contemplated that the inner surface portion 314 may comprise a variety of other shapes, such as a partial circular (as illustrated in FIG. 2), oval, flat or irregular shape. In addition, it is also contemplated that the shell 302 may comprise other features. For example, similar to the illustration in FIG. 3, it may comprise an internal ring, which may prevent the pillar 308 from exerting excessive force against the damping sleeve 310.

The shell 302 may comprise any biocompatible material, such as 22Co-13Cr-5Mo, cobalt chrome, stainless steel, titanium, shape memory alloys, polymers, carbon fiber, polythene, polyurethane, polyethylene porous material or silicone.

In furtherance of this example, the damping sleeve 310 is coupled with the shell 302. The damping sleeve 310 may serve to prevent the pillar 308 from moving too far from its designed position and become dislocated. In addition, the damping sleeve 308 may provide various degrees of flexibility for the disc replacement device, and modulates the stiffness of the disc replacement device. As described previously, the damping sleeve 308 may be mated with the shell 302 in a variety of means.

In this illustration, from a cross-sectional view, the damping sleeve 308 has an inner surface 318 that may comprise a variety of shapes, such as a partial rectangular, partial circular, oval, flat or irregular shape.

The damping sleeve 318 has a width W3, while the coupling portion of the shell 302 has a width W4. To accommodate the movement of a spine, the width W4 may be larger than the width W3 to allow mobility of the damping sleeve 308 relative to the shell 302. However, it is also contemplated that the W4 may be nearly identical to W3, so that a tight fit may be provide between the damping sleeve 308 and the shell 302.

It is contemplated that the disc replacement devices disclosed in this invention may be provided in different sizes to accommodate the desired disc space. For example, a disc replacement device for the lumber area may be larger than a disc replacement device for the neck area.

Utilization of the present invention will now be briefly described. It will be understood that access to the disc space, disc removal, and end plate preparation are known in the art and will be only briefly described herein. For example, procedures and instruments useable in a posterior approach to the disc space are disclosed in U.S. Pat. No. 6,241,729 (assigned to SDGI Holdings, Inc.), and a publication by Sofamor Danek© 1996 entitled “Surgical Technique using Bone Dowel Instrumentation for Posterior Approach”, each of which is incorporated herein by reference in its entirety.

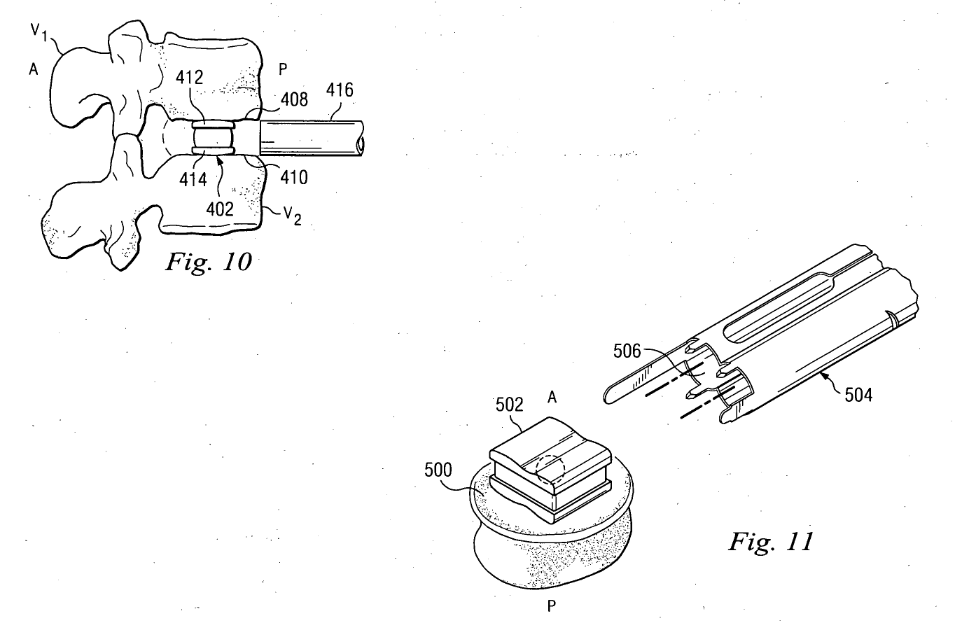

Referring now to FIGS. 9 and 10, in one embodiment, a disc space 406 is positioned between an upper vertebral body V1 and a lower vertebral body V2. The anterior side of the vertebral bodies is indicated by the letter “A”, and their posterior side is indicated by the letter “P”. Two disc replacement devices 402 and 404 are inserted into the disc space 406. It will be understood that a fewer or greater number of disc replacement devices, each of which may be any of those disc replacement devices described previously, may be utilized in the disc space 406.

Insertion preparation may be made by removing material from the disc space 406 and forming, by reaming, cutting, tapping or other technique, a portion 408 in the upper vertebral body V1 that is suitable for receiving an upper shell of the disc replacement device 402. In procedures utilizing an insertion sleeve, a laminectomy may also be performed through the sleeve. Similarly, a corresponding and, aligned portion 410 is formed in the lower vertebral body V2. The disc replacement device, 402 may then be inserted with an upper shell 412 contacting and/or engaging the portion 408, and a lower shell 414 contacting and/or engaging the portion 410.

As shown in FIGS. 9-10, portions of bony material can remain anteriorly and posteriorly of a disc replacement device 402 to countersink the disc replacement device 402 in the disc space 406 and further resist its expulsion from the disc space 406. A variety of procedures, including a posterior approach to the disc space 406, may be employed to implant the disc replacement devices 402 and 404 into the disc space 406. Further, the insertion may be accomplished by utilizing a single-barrel tube or insertion sleeve 416 via pushing or threading the disc replacement devices 402 and 404 into position through the single-barrel tube or insertion sleeve 416. By inserting two disc replacement devices 402 and 404 in the disc space 406, each of them will act independently to provide three degrees of motion, while the upper and lower shells will protect the balls from excessive wear or expulsion.

Referring now to FIG. 11, in one embodiment, a disc space 500 between vertebral bodies is configured for a disc replacement device 502 insertion by utilizing a double-barrel insertion sleeve 504. In operation, the insertion procedure is performed by an anterior approach to the disc space 500. Procedures and instruments useable in an anterior approach are disclosed in U.S. Pat. No. 6,428,541 (assigned to SDGI Holdings, Inc.), and a publication by Sofamor Danek© 1996 entitled “Surgical Technique using Bone Dowel Instrumentation for Anterior Approach”, each of which is incorporated herein by reference in its entirety.

An interior channel 506 of the insertion sleeve 504 and the disc replacement device 502 may be sized, so that the disc replacement device 502 is maintained in a partially compressed condition during insertion. It will be understood that the endplates of the adjacent vertebral body to the disc space 500 are prepared to receive the disc replacement device 504 prior to its insertion. Techniques for shaping vertebral body endplates to conform them to the geometry of devices positioned in the disc space are well-known in the art and will not be further described herein. In one embodiment, the locations for the shells of the disc replacement device 502 are prepared by reaming the disc space 500, and that the reamed disc space 500 will allow the disc replacement device 502 to be countersunk in the disc space 500 to prevent its expulsion from the disc space 500.

It is also contemplated that the disc replacement device 502 may be inserted by a lateral approach or other methods.

The present invention contemplates providing a variety of shells, damping sleeves, balls/pillars to achieve the necessary adaptation of a disc replacement device into a disc space between vertebral bodies while taking into consideration a surgeon's access to a disc space. Even though the combinations have been disclosed herein as being applicable to a particular disc space, this is not a limitation on the use of such devices, and uses in other manners or other disc space is contemplated as being within the spirit of the present invention.

Although only a few exemplary embodiments of this invention have been described above in details, those skilled in the art will readily appreciate that many modifications are possible in the exemplary embodiments without materially departing from the novel teachings and advantages of this invention. Also, features illustrated and discussed above with respect to some embodiments can be combined with features illustrated and discussed above with respect to other embodiments. Accordingly, all such modifications are intended to be included within the scope of this invention.

1. A disc replacement device comprising a shell, a fulcrum, and a damping sleeve, wherein the shell comprises:
a first portion adapted for articulating with the fulcrum; and
a second portion adapted for coupling with the damping sleeve.

2. The disc replacement device of claim 1 wherein the fulcrum is a spherical ball bearing.

3. The disc replacement device of claim 1 wherein the first portion comprise a flat surface.

4. The disc replacement device of claim 2 wherein the first portion comprises a concave surface.

5. The disc replacement device of claim 1 wherein the first portion comprises an irregular surface.

6. The disc replacement device of claim 1 wherein the damping sleeve is configured to provides flexibility between the first and second shell portions.

7. The disc replacement device of claim 1 wherein the damping sleeve comprises varied thickness.

8. The disc replacement device of claim 1 wherein the shell comprises a metal substance.

9. The disc replacement device of claim 1 wherein the shell comprises shape memory alloys.

10. The disc replacement device of claim 1 wherein the shell comprises an orthopedic articular bearing material.

11. The disc replacement device of claim 1 wherein the damping sleeve comprises silicone.

12. The disc replacement device of claim 1 wherein the damping sleeve comprises shape memory alloys.

13. The disc replacement device of claim 1 wherein the damping sleeve is configured to produce a cavity for receiving a lubrication medium.

14. The disc replacement device of claim 1 further comprising an internal ring.

15. A shell system for use with a spherical ball bearing disc replacement device, the shell system comprising:
a first shell comprising a first portion adapted for coupling with a second shell and a second portion adapted for coupling with a damping sleeve; and
a second shell comprising a first surface adapted for coupling with the first portion of the first shell and a second surface adapted for articulating with the spherical ball bearing.

16. The shell system of claim 15 wherein the first shell comprises titanium.

17. The shell system of claim 15 wherein the second shell comprises at least one from the group consisting of ceramic, cobalt chrome, polymer, stainless steel, and polyethylene.

18. The shell system of claim 15 further, comprising an internal ring.

19. A disc replacement device, comprising:
a first shell comprising an opening and an inner surface portion;
a pillar adapted for coupling with the first shell at the opening; and
a damping sleeve for coupling with the first shell at the inner surface.

20. The disc replacement device of claim 19 wherein the pillar comprises at least one shape memory alloy.

21. The disc replacement device of claim 19 further comprising:
a spherical shaped device positioned proximate to the first shell.

22. The disc replacement device of claim 19 further comprising an internal ring.
